(19) United States
(12) Patent Application Publication
(10) Pub. No.: US 2025/0285711 A1
(43) Pub. Date: Sep. 11, 2025

(54) Functional Genomic Imaging Method, Electronic Device and Medium

(71) Applicant: Xiaorong Gu

(72) Inventor: Xiaorong Gu

(21) Appl. No.: 18/597,503

(22) Filed: Mar. 6, 2024

(51) Int. Cl. G16B 45/00; G16B 25/10; G16B 40/20

(52) U.S. Cl. CPC G16B 45/00; G16B 25/10; G16B 40/20

(57) ABSTRACT

The present disclosure discloses a Functional Genomic Imaging method. Genes are filtered based on the expression levels and other information first to improve the ratio of effective genes, and then a correlation matrix of gene co-expression is generated based on the filtered gene data to obtain a co-expression network; data conversion is performed based on the correlation matrix of gene co-expression to obtain a distance matrix; and finally, the co-expression network is subjected to dimensionality reduction and cluster analysis, thus obtaining the visualized gene expression according to the analysis result. The Functional Genomic Imaging method provided by the present disclosure achieves a better visualization effect of the genetic co-expression network and can obtain a co-expressed gene cluster more effectively.

### CROSS-REFERENCE TO RELATED APPLICATION

This application claims priority from the Chinese patent application 202310211758.9 filed Mar. 7, 2023, the content of which is incorporated herein in the entirety by reference.

### TECHNICAL FIELD

The present disclosure relates to the technical field of RNA sequencing, and in particular to a Functional Genomic Imaging method, an electronic device and a medium.

### BACKGROUND

RNA sequencing, namely, RNA-Seq, refers to a technique of sequencing analysis by high-throughput sequencing technique, thus reflecting the expression level of mRNA, smallRNA, noncoding RNA and the like. Transcriptome is a set of all the transcripts produced by a certain species or a particular cell type. RNA-Seq can be applied to research genetic functions and structures at overall level, so as to reveal the molecular mechanism of a particular biological process and course of diseases. RNA-Seq has been widely applied to fields such as basic research, clinical diagnosis, research and development of drugs.

However, RNA data contains excessively enormous information. Therefore, how to dig out, understand and visually display the information of RNA data fully becomes an important problem to be solved in the industry.

### SUMMARY

Directed to partial or total problems in the prior art, in a first aspect, the present disclosure provides a Functional Genomic Imaging (FGI) method, including:
filtering genes based on expression level to improve a ratio of effective genes;
generating a correlation matrix of gene co-expression based on the filtered gene data to obtain a co-expression network;
performing data conversion based on the correlation matrix of gene co-expression to obtain a distance matrix of the gene; and
performing dimensionality reduction and cluster analysis based on the distance matrix, and obtaining visualized gene expression according to an analysis result.

Further, the step of filtering genes includes a step of: eliminating a data of non-coding genes and particular housekeeping genes from the gene data.

Further, the data of particular housekeeping genes includes mitochondrial and ribosomal genes.

Further, the correlation matrix of gene co-expression is obtained via Weighted Gene Co-expression Network Analysis (WGCNA).

Further, the dimensionality reduction and cluster analysis include:
performing dimensionality reduction on the co-expression network with a tSNE descending dimension method based on the distance matrix; and
performing cluster analysis on the distance matrix with an improved PhenoGraph clustering algorithm to obtain a co-expressed gene cluster, useful for subsequent functional annotation.

Further, the Functional Genomic Imaging method further includes:
performing calibration on a sample to be analyzed first prior to filtering the sample to be analyzed.

Further, the calibration includes:
performing inter-batch calibration on the sample to be analyzed by ComBat-seq, and
performing calibration on a library size of the sample after the inter-batch calibration via EdgeR to obtain CPM data.

Further, the step of obtaining visualized gene expression according to an analysis result includes:
calculating a Z-Score value of each gene in a normal tissue and a tumor tissue to form an FGI gene scatter plot.

Further, the step of obtaining visualized gene expression according to an analysis result further includes:
performing a local statistic on each gene expression data in the FGI gene scatter plot to form an FGI gene cloud map.

Further, the step of obtaining visualized gene expression according to an analysis result further includes:
highlighting a Geneset correlated to cell functions from a public database in the FGI gene cloud map.
according to the Functional Genomic Imaging method mentioned above, in a second aspect, the present disclosure provides an electronic device for use in Functional Genomic Imaging, including a memory and a processor, where the memory is configured to store a computer program that executes the Functional Genomic Imaging method mentioned above when running on the processor.

In a third aspect, the present disclosure provides a computer readable storage medium for Functional Genomic Imaging, where a computer program is stored therein, and the computer program executes the Functional Genomic Imaging method mentioned above when running on the processor.

In the Functional Genomic Imaging method provided by the present disclosure, the quantity of effective genes in a co-expression network is improved via filtering, and then correlated dimension reduction and clustering algorithm are introduced for dimension reduction and cluster analysis on a correlation network, thus achieving a better visualization effect of the genetic co-expression network, obtaining co-expressed gene clusters more effectively and improving the efficiency of gene sequencing. Moreover, the method further includes a process of effective data calibration to calibrate the batch effect between the sequencing sample and public database, thus ensuring the stability and accuracy of data interpretation.

### BRIEF DESCRIPTION OF THE DRAWINGS

To further describe the above advantages and features, and other advantages in the examples of the present disclosure, examples of the present disclosure will be described more specifically with reference to the accompanying drawings below. It is appreciable that these accompanying drawings are merely illustrative of typical examples of the present disclosure, but are not construed as limiting the scope of the protection thereof. For the purpose of clear and definite description, the same or corresponding parts are denoted with same or similar numerals in the accompanying drawings.

FIG. 1 is a process diagram showing a Functional Genomic Imaging method in an example of the present disclosure;

FIG. 2 shows an FGI gene scatter plot of a certain cancer obtained using the Functional Genomic Imaging method in an example of the present disclosure;

FIGS. 3a-3c show highlights, gene quantity statistics and positions of gene clusters of Genesets from the public database in the FGI gene scatter plot; and

FIG. 4a-4d show FGI gene scatter plots and FGI gene cloud maps of two different samples obtained by the Functional Genomic Imaging method in an example of the present disclosure, respectively.

### DETAILED DESCRIPTION OF THE EMBODIMENTS

The present disclosure will be described with reference to the examples below. However, a person skilled in the art will realize that each example may be implemented without one or more peculiar details or in combination with other replacements and/or additional methods. In other cases, structures or operations known to the public are not shown or described in detail to avoid obscuring the idea of the present disclosure. Similarly, for the purpose of explanation, specific quantity and configuration are set forth here to provide a full understanding to the examples of the present disclosure. However, the present disclosure is not limited to these specific details. Moreover, it should be understood that examples as shown in the accompanying drawings are illustrative and are not always drawn according to right proportions.

In this description, the citation of “an example” or “the example” means that specific features, structures or properties descried in combination with the examples are included in at least one example of this present disclosure. The phrase “in an example” throughout the description not always refers to the same example.

It should be indicated that examples in this present disclosure are illustrative of the steps of the method according to a specified order, but this is only for the description of the detailed example, but not construed as limiting the order of the steps. On the contrary, the order of steps may be adjusted according to the actual demands in different examples of the present disclosure.

Each module of the system according to the present disclosure may be achieved by software, hardware, firmware or a combination thereof. When the module is achieved with software, functions of the module may be achieved by the computer program flow, for example, a module may be achieved via a code segment (e.g., code segments of C, C++ and the like) stored in a memory device (e.g., hard disk, internal storage, and the like); when the code segment is executed by a processor, corresponding functions of the module may be achieved. When the module is achieved via hardware, functions of the module may be achieved by setting corresponding hardware structures, for example, by hardware programming with a field-programmable gate array (FPGA) and other programmable devices, or by designing an application-specific integrated circuit (ASIC) including a plurality of electron devices such as, transistors, resistors and capacitors. When the module is achieved via firmware, functions of the module may be written into read-only memories such as, EPROM or EEPROM of a device in a form of procedure code. Moreover, when the procedure code is executed by a processor, corresponding functions of the module may be achieved. Furthermore, certain functions of a module may be achieved via a separate hardware or achieved in cooperation with the hardware, for example, the detection function is achieved via a corresponding sensor (e.g., a proximity transducer, an acceleration sensor, a gyroscope, and the like); the signal emission function is achieved via a corresponding communication device (e.g., a Bluetooth device, an infrared communication device, a baseband communication device, a Wi-Fi communication device, and the like); the output function is achieved via a corresponding output device (e.g., a display, a loudspeaker, and the like), and so on.

To provide a basis for subtyping determination and single-sample sequencing data of cancer, a gene co-expression network may be built based on TCGA and other public databases, and then subjected to visualized analysis, thus obtaining a series of co-expressed gene clusters among samples of a specific type of cancer. Moreover, annotation is performed according to the gene function enrichment. Meanwhile, the expression quantity, expression range and other data of each gene in the population of cancer samples and the population of normal tissue samples are integrated. To improve the detection efficiency and flexibility of the co-expression network, the present disclosure achieves optimization based on the existing Functional Genomic Imaging (FGI). The co-expression neural network is subjected to dimensionality reduction and cluster analysis with an improved algorithm first to achieve a better visualization effect of the genetic co-expression network, thus obtaining co-expressed gene clusters more effectively. Secondly, a process of effective data calibration is established to calibrate the batch effect between the sequencing sample and public database, thus ensuring the stability and accuracy of data interpretation.

Technical solutions of the present disclosure are further described with reference to the accompanying drawings of the example below.

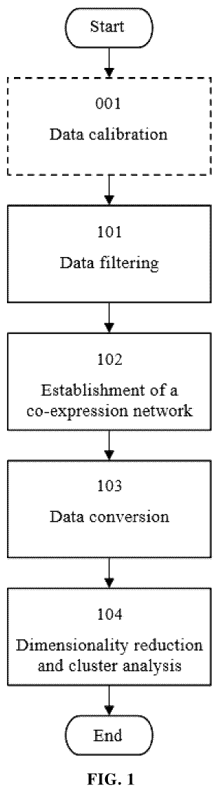

FIG. 1 is a process diagram showing a Functional Genomic Imaging method in an example of the present disclosure. As shown in FIG. 1, a Functional Genomic Imaging method includes:

Firstly, data was filtered in the step 101. Prior to the establishment of the co-expression network, genes were filtered first according to the expression level and other information to improve the quantity of effective genes in the co-expression network to the greatest extent. Prior to the establishment of the co-expression network, non-coding genes and partial housekeeping genes were first filtered out from the genes to increase the quantity of effective genes incorporated into the co-expression network such that the obtained FGI gene co-expression model has higher representativeness. In an example of the present disclosure, the non-coding genes include pseudo genes. In a further example of the present disclosure, the housekeeping genes filtered out mainly refer to genes related to mitochondria and ribosome.

The co-expression network was then established in the step 102. A correlation matrix of gene co-expression was generated based on the filtered gene data to obtain the co-expression network. In an example of the present disclosure, the correlation matrix of gene co-expression was obtained via Weighted Gene Co-expression Network Analysis (WGCNA). WGCNA is a kind of method to analyze gene expression patterns of multiple samples; the method may cluster the genes with similar expression pattern and analyze a correlation between the module and a particular character or phenotype. Firstly, the genetic network was assumed to obey scale-free distribution; the correlation matrix of gene co-expression and adjacency functions of the genetic network were defined, and then coefficients of variation of different nodes were calculated, and a hierarchical clustering tree was established based thereon. Different branches of the hierarchical clustering tree represent different gene modules; and gene modules include genes with similar expression patterns.

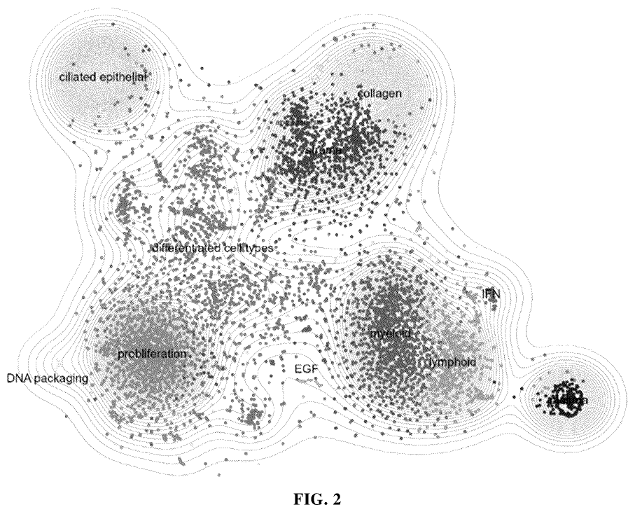

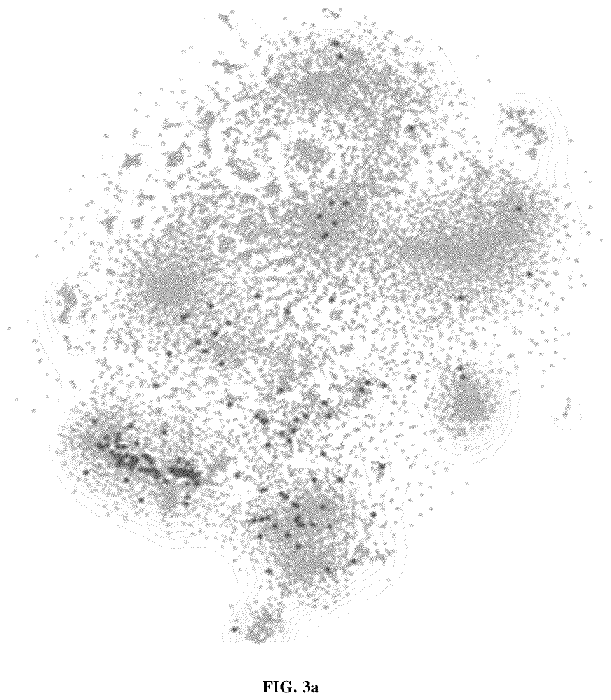

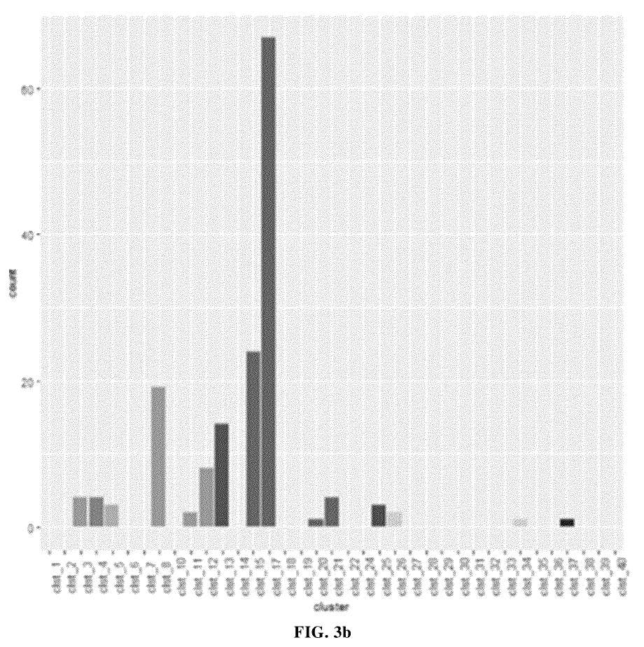

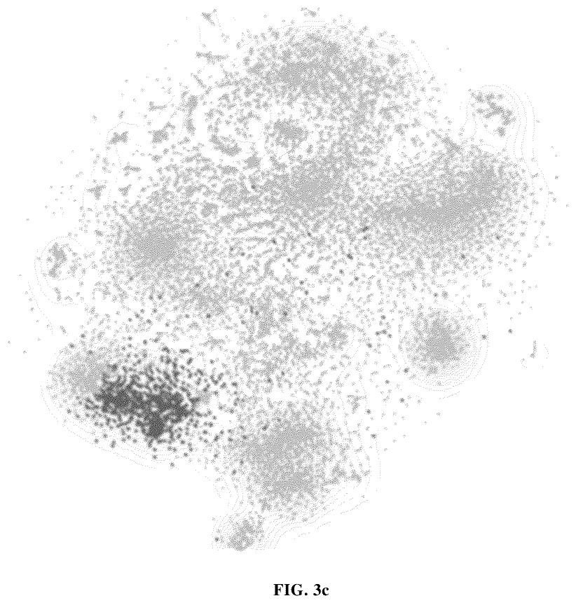

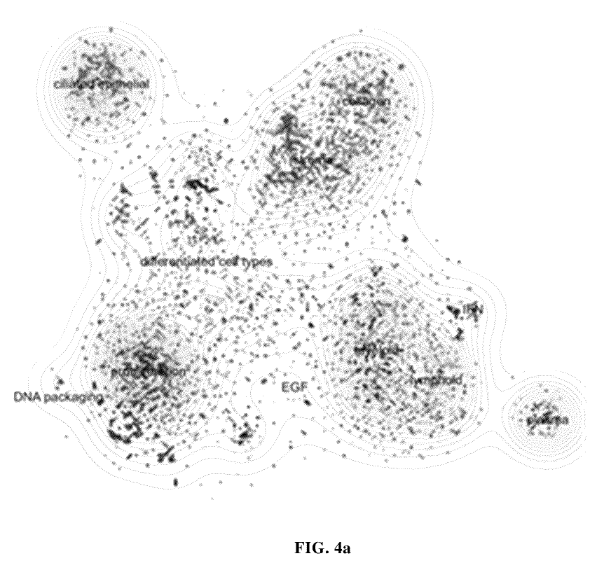

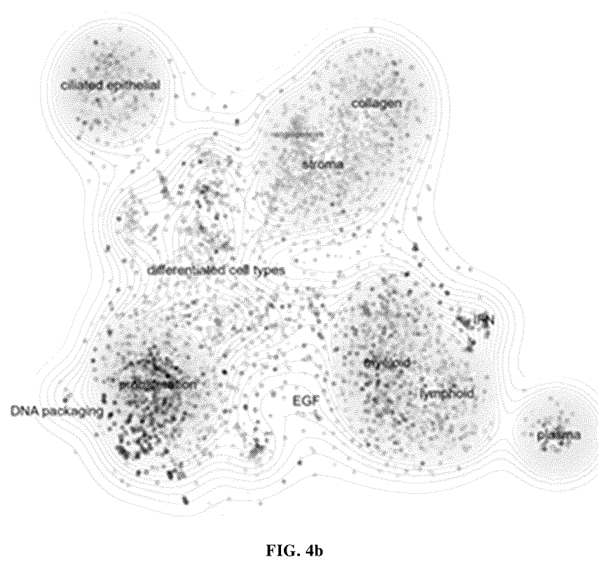

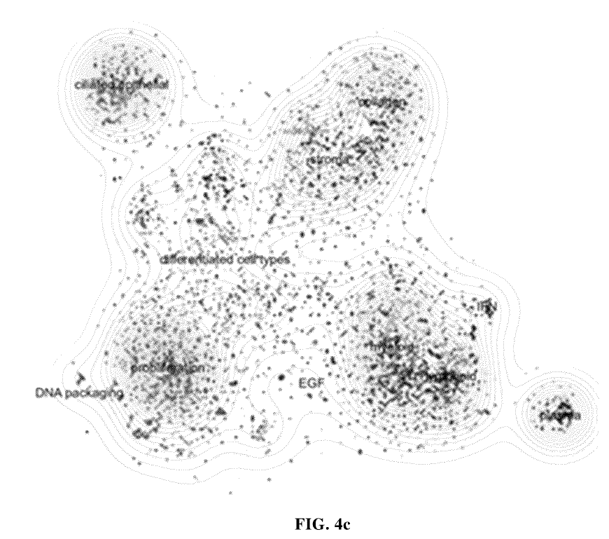

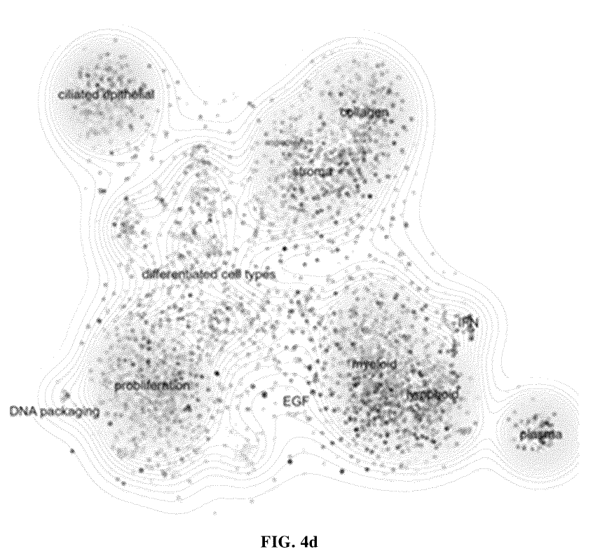

Data conversion was subsequently performed in the step 103. Data conversion was performed based on the correlation matrix of gene co-expression obtained in the step 102 to obtain a distance matrix of gene; and
dimensionality reduction and cluster analysis were performed finally in the step 104. Dimensionality reduction and cluster analysis were performed based on the distance matrix, and visualized gene expression was obtained according to an analysis result. In an example of the present disclosure, an improved PhenoGraph clustering algorithm was newly introduced for cluster analysis on the distance matrix, which improves the detection efficiency and flexibility of the co-expressed module and effectively improves the quantity of effective genes in the co-expression network. Moreover, the co-expression network is visualized via the optimized dimensionality reduction algorithm such that the gene clusters/co-expressed modules are displayed more visually. Specifically, in an example of the present disclosure, the co-expression network was subjected to dimensionality reduction with a tSNE descending dimension method based on the distance matrix; the distance expression was subjected to cluster analysis with the improved PhenoGraph clustering algorithm to obtain co-expressed gene cluster, for use in the subsequent functional annotation, namely, obtaining the FGI gene scatter plot and the gene clusters correlated to a specific type of cancer. In an example of the present disclosure, relative expression level of the genes and correlated Genesets in the co-expression network were displayed visually with the FGI gene scatter plot and the FGI gene cloud map. The FGI gene scatter plot is useful for displaying the Z-Score value of each gene obtained by calculation in normal tissues and tumor tissues. The Z-Score value may visually show the degree of gene expression level of each functional cluster. FIG. 2 shows an FGI gene scatter plot of a certain cancer obtained using the Functional Genomic Imaging method in an example of the present disclosure. As shown in the figure, different gray scale points show the gene clusters obtained via clustering, which relates to multiple aspects such as proliferation, immunization, Stroma and differentiation. The FGI gene cloud map shows local statistics on each genetic expression data in the FGI gene scatter plot, so as to exclude the influences caused by mutual covering of scatter points in the FGI gene scatter plot. To visually display the co-expression correlation of the genes in the Geneset and the correlation with the gene clusters in the existing model thereof, in an example of the present disclosure, Genesets correlated to cell functions from public databases such as, MSigDB and Reactome may be further highlighted in the FGI gene scatter plot. FIGS. 3a-3c show highlights, gene quantity statistics and positions of gene clusters of Genesets from the public database in the FGI gene scatter plot. The dark-color points in FIG. 3a show genes contained in Geneset; FIG. 3b shows quantity statistics of the genes falling within each gene cluster; and FIG. 3c shows positions of the gene clusters involved, showing that the gene clusters are greatly correlated to Geneset. FIGS. 4a-4d show FGI gene scatter plots and FGI gene cloud maps of two different samples obtained by the Functional Genomic Imaging method in an example of the present disclosure, respectively. FIG. 4a shows an FGI gene cloud map of the sample 1; FIG. 4b shows an FGI gene scatter plot of the sample 1; FIG. 4c shows an FGI gene cloud map of the sample 2; FIG. 4d shows an FGI gene scatter plot of the sample 2. As can be seen from the figures, the gene clusters correlated to proliferation have a higher expression level in the sample 2; the gene clusters correlated to stroma and angiogenesis have a higher expression level in the sample 1.

Due to the differences in many aspects such as, base-building method and sequencing platform, there probably exists batch effect between the test sample and the public database. The existence of the batch effect will significantly affect the judgment on the gene expression level. To solve such a problem, in an example of the present disclosure, a step 001, data calibration, may be further performed prior to the step 101:

In the step 001, the sample to be analyzed was subjected to inter-batch calibration by ComBat-seq, and the Library Size of the sample after inter-batch calibration was calibrated by EdgeR, thus obtaining CPM data, useful for the subsequent analysis of the co-expression network.

Based on the FGI method mentioned above, the present disclosure further provides a visual analysis method for a single sample, including:
sequencing data was calibrated to amend the batch effect between the sample and public database;
expression of each gene in the co-expression network was displayed using the FGI gene scatter plot and FGI gene cloud map;

Since single-gene expression will be affected by some random factors, the expression situation of each co-expressed gene cluster and clinical subtype-correlated Geneset may be analyzed comprehensively to obtain the information in the aspects such as, tumor proliferation vitality, differentiation level, angiogenesis, Stroma composition and immune activity; and
reaction conditions of a portion of drugs were predicted according to the immunophenotyping of the sample, and target expression level of the approved drugs and other information.

In this present disclosure, a visual analysis process based on co-expression network is established to organically integrate the gene co-expression information, clinical prognosis Geneset, drug targets and other information. On the one hand, the present disclosure shows the information of tumor samples in the aspects such as, proliferation vitality, differentiation level, angiogenesis, Stroma composition, immune activity, and signal pathway, on the other hand, improves the readability of data analysis report to a great extent, which provides convenience for the application of the sequencing technique in clinical aspect.

According to the Functional Genomic Imaging method mentioned above, in a second aspect, the present disclosure provides an electronic device for use in Functional Genomic Imaging, including a memory and a processor, where the memory is configured to store a computer program that executes the Functional Genomic Imaging method mentioned above when running on the processor.

In a third aspect, the present disclosure provides a computer readable storage medium for Functional Genomic Imaging, where a computer program is stored therein, and the computer program executes the Functional Genomic Imaging method mentioned above when running on the processor.

Even though examples of the present disclosure are described above, it should be appreciated that they are merely used as examples, but are not construed as limiting the scope of the protection. It is apparent for a person skilled in the art to make various combinations, modifications and alterations within the spirit and scope of the present disclosure. Therefore, the scope of the protection of the present disclosure disclosed herein shall be not limited to the exemplary examples disclosed above, but defined according to the claims attached and equivalent substitutions thereof only.

1. A Functional Genomic Imaging method, comprising the steps of:
filtering genes based on expression level to improve a ratio of effective genes;
generating a correlation matrix of gene co-expression based on the filtered gene data to obtain a co-expression network;
performing data conversion based on the correlation matrix of gene co-expression to obtain a distance matrix of gene; and
performing dimensionality reduction and cluster analysis based on the distance matrix, and obtaining visualized gene expression according to an analysis result.

2. The Functional Genomic Imaging method according to claim 1, wherein the step of filtering genes comprises a step of:
clearing a data of non-coding genes and particular housekeeping genes.

3. The Functional Genomic Imaging method according to claim 2, wherein the data of particular housekeeping genes comprises mitochondrial and ribosomal genes.

4. The Functional Genomic Imaging method according to claim 1, wherein the correlation matrix of gene co-expression is obtained via Weighted Gene Co-Expression Network Analysis.

5. The Functional Genomic Imaging method according to claim 1, wherein the step of dimensionality reduction and cluster analysis based on the distance matrix comprises steps of:
performing dimensionality reduction on the co-expression network with a tSNE descending dimension method based on the distance matrix; and
performing cluster analysis on the distance matrix with an improved PhenoGraph clustering algorithm to obtain co-expressed gene clusters, useful for subsequent functional annotation.

6. The Functional Genomic Imaging method according to claim 1, further comprising a step of:
performing calibration on a sample to be analyzed first prior to filtering the sample to be analyzed.

7. The Functional Genomic Imaging method according to claim 6, wherein the calibration comprises:
performing inter-batch calibration on the sample to be analyzed by ComBat-seq, and
performing calibration on a library size of the sample after inter-batch calibration via EdgeR.

8. The Functional Genomic Imaging method according to claim 1, wherein the step of obtaining visualized gene expression according to an analysis result comprises:
calculating a Z-Score value of each gene in a normal tissue and a tumor tissue to form an FGI gene scatter plot.

9. The Functional Genomic Imaging method according to claim 8, wherein the step of obtaining visualized gene expression according to an analysis result further comprises:
performing a local statistic on each gene expression data in the FGI gene scatter plot to form an FGI gene cloud map.

10. The Functional Genomic Imaging method according to claim 8, wherein the step of obtaining visualized gene expression according to an analysis result further comprises:
highlighting a Geneset correlated to cell functions from a public database in the FGI gene cloud map.

11. An electronic device for use in Functional Genomic Imaging,
comprising a memory and a processor, wherein the memory is configured to store a computer program, and the computer program executes the Functional Genomic Imaging method of claim 1 when running on the processor.

12. A computer readable storage medium for Functional Genomic Imaging, wherein a computer program is stored therein, and the computer program executes the Functional Genomic Imaging method of claim 1 when running on the processor.

13. The electronic device of claim 11, wherein the step of filtering genes comprises a step of:
clearing a data of non-coding genes and particular housekeeping genes.

14. The electronic device of claim 13, wherein the data of particular housekeeping genes comprises mitochondrial and ribosomal genes.

15. The electronic device of claim 11, wherein the correlation matrix of gene co-expression is obtained via Weighted Gene Co-Expression Network Analysis.

16. The electronic device of claim 11, wherein the step of dimensionality reduction and cluster analysis based on the distance matrix comprises steps of:
performing dimensionality reduction on the co-expression network with a tSNE descending dimension method based on the distance matrix; and
performing cluster analysis on the distance matrix with an improved PhenoGraph clustering algorithm to obtain co-expressed gene clusters, useful for subsequent functional annotation.

17. The electronic device of claim 11, further comprising a step of:
performing calibration on a sample to be analyzed first prior to filtering the sample to be analyzed.

18. The electronic device of claim 17, wherein the calibration comprises:
performing inter-batch calibration on the sample to be analyzed by ComBat-seq, and
performing calibration on a library size of the sample after inter-batch calibration via EdgeR.

19. The electronic device of claim 11, wherein the step of obtaining visualized gene expression according to an analysis result comprises:
calculating a Z-Score value of each gene in a normal tissue and a tumor tissue to form an FGI gene scatter plot.

20. The electronic device of claim 19, wherein the step of obtaining visualized gene expression according to an analysis result further comprises:
performing a local statistic on each gene expression data in the FGI gene scatter plot to form an FGI gene cloud map.
